(19) United States
(12) Patent Application Publication
(10) Pub. No.: US 2006/0014699 A1
(43) Pub. Date: Jan. 19, 2006

(54) Aqueous pharmaceutical formulaton comprising the thrombin inhibitor melagatran and use of the formulation in the manufacture of a medicament for use by nasal administration in treating thromboembolism

(75) Inventors: Peter Edman, Molndal (SE); Karin Wingstrand, Molndal (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/533,869

(22) PCT Filed: Nov. 11, 2003
(86) PCT No.: PCT/SE03/01738

§ 371 Date: May 5, 2005

(30) Foreign Application Priority Data
Nov. 12, 2002 (SE) 0203349-6

(51) Int. Cl. A61K 38/04

(52) U.S. Cl. 514/19

(57) ABSTRACT

An aqueous pharmaceutical formulation comprising the thrombin inhibitor HOOC—CH₂—(R)-Cgl-Aze-Pab (melagatran), or a pharmaceutically-acceptable derivative thereof, a process for the preparation of such a pharmaceutical formulation, the use of such a formulation in the treatment of thromboembolism as well as a method of treating a patient in need of antithrombotic treatment and thromboembolism by using said formulation via nasal administration.

The present invention relates to an aqueous pharmaceutical formulation comprising the thrombin inhibitor HOOC—CH₂—(R)-Cgl-Aze-Pab (hereinafter melagatran) or a pharmaceutically-acceptable derivative thereof, the use of such a formulation in the treatment of thromboembolism, as well as a method of treating a patient in need of such a treatment by using said formulation, via a particular route of administration.

Blood coagulation is the key process involved in both haemostasis (i.e. prevention of blood loss from a damaged vessel) and thrombosis (i.e. the pathological occlusion of a blood vessel by a blood clot). Coagulation is the result of a complex series of enzymatic reactions; one of the final steps is conversion of the proenzyme prothrombin to the active enzyme thrombin.

Thrombin plays a central role in coagulation. It activates platelets, it converts fibrinogen into fibrin monomers, which polymerize spontaneously into filaments, and it activates factor XIII, which in turn crosslinks the polymer to insoluble fibrin. Thrombin further activates factor V and factor VIII in a positive feedback reaction. Inhibitors of thrombin are therefore expected to be effective anticoagulants by inhibition of platelet activation, fibrin formation and fibrin stabilization. By inhibiting the positive feedback mechanism such inhibitors are expected to exert inhibition early in the chain of events leading to coagulation and thrombosis. Melagatran is a thrombin inhibitor in active development.

Peptidic or peptide like thrombin inhibitors, like many other peptide-like substances, are prone to limited absorption when administered. This may be due to the influence of different barriers of metabolic and physical character, such as enzymatic degradation, tendencies toward complex formation with components from the formulation or the biological environment, limitations in epithelial transport etc.

In seeking desirable absorption and a favourable pharmaco-kinetic profile for an active compound, many different administration routes are possible, such as oral, rectal, buccal, nasal, pulmonary, inhalation route etc., and are disclosed, for example in WO 96/16671 (U.S. Pat. No. 5,795,896) which specifically concerns formulations of melagatran.

Additionally, it may be necessary to administer pharmaceutically active compounds frequently throughout the day in order to maintain a desired therapeutic level of active principle in plasma and/or body tissues. This is particularly the case where it is intended to deliver a uniform response over an extended period of time, and the most common routes of administration used are oral and parenteral. However, the parenteral route can be inconvenient, and oral administration can result in unacceptably low bioavailabilities.

Nasal delivery is a feasible alternative to oral or parenteral administration for some drugs, although many factors may influence the permeability of nasal mucosa to different compounds and such administration is often less attractive. Potential advantages of nasal administration are high permeability of the nasal epithelium and, as a result of the rather large surface area of the nasal cavity and the relatively high blood flow, rapid absorption. Furthermore, self-medication is easy and convenient.

One object of the present invention is to provide pharmaceutical formulations comprising the thrombin inhibitor HOOC—CH₂—(R)-Cgl-Aze-Pab (melagatran), or a pharmaceutically-acceptable derivative thereof, which are suitable for administration via the nasal route, and which deliver attractive absorption characteristics and a favourable pharmaco-kinetic profile.

In order to achieve suitable absorption, many different formulations of this therapeutically active drug are possible. For example, WO 96/16671 discusses the use of absorption enhancing agents, such as, but not limited to, surface active agents, chelating agents, lipids, other drugs and polymers to obtain positive effects which result in an enhanced and/or less variable absorption of the therapeutically active agent.

We have studied the use of several absorption enhancing agents in nasal administration (see Experimental Section) which confirm the improved absorption disclosed in WO 96/16671.

However, despite these results, a limiting factor associated with the addition of enhancers to a formulation for nasal administration is the potential toxicity to the nasal mucosa Nasal absorption enhancers are required to be non-irritating, non-toxic and non-allergenic or at least to have immediately reversible effects. Moreover, they should be potent, compatible with the drug and other excipients in the formulation and systemically inert in the concentrations used. Potential enhancers have to be carefully evaluated to be acceptable in their enhancing ability and overall safety profile, with regard to both local and systemic effects.

With these potential drawbacks in mind the development of nasal formulations would not appear attractive. This is particularly so for anticoagulant compounds such as melagatran, which might potentially lead to undesirable, or uncontrolled, bleeding in the sensitive nasal cavity.

However, as the results in the Experimental Section for healthy male humans demonstrate, it has now been found that the nasal administration of the therapeutically active thrombin inhibitor HOOC—CH₂—(R)-Cgl-Aze-Pab is particularly attractive in pharmaceutical formulations containing said therapeutically active compound, but without the use of additional absorption enhancers.

Accordingly, in one aspect of invention we provide an aqueous pharmaceutical formulation comprising the thrombin inhibitor HOOC—CH₂—(R)-Cgl-Aze-Pab (melagatran), or a pharmaceutically-acceptable derivative thereof, for use by nasal administration.

In another aspect we provide an aqueous pharmaceutical formulation comprising the thrombin inhibitor HOOC—CH₂—(R)-Cgl-Aze-Pab (melagatran), or a pharmaceutically-acceptable derivative thereof, for use by nasal administration in antithrombotic treatment.

In another aspect we provide an aqueous pharmaceutical formulation comprising the thrombin inhibitor HOOC—CH₂—(R)-Cgl-Aze-Pab (melagatran), or a pharmaceutically-acceptable derivative thereof, for use by nasal administration in treating thromboembolism.

Further aspects of the invention include:—

The use of an aqueous pharmaceutical formulation comprising the thrombin inhibitor HOOC—CH₂—(R)-Cgl-Aze-Pab (melagatran), or a pharmaceutically-acceptable derivative thereof, in the manufacture of a medicament for use by nasal administration in antithrombotic treatment.

The use of an aqueous pharmaceutical formulation comprising the thrombin inhibitor HOOC—CH₂—(R)-Cgl-Aze-Pab (melagatran), or a pharmaceutically-acceptable derivative thereof, in the manufacture of a medicament for use by nasal administration in treating thromboembolism.

A method of treating a patient in need of antithrombotic treatment by nasally administering an aqueous pharmaceutical formulation comprising the thrombin inhibitor HOOC—CH₂—(R)-Cgl-Aze-Pab (melagatran), or a pharmaceutically-acceptable derivative thereof.

A method of treating thromboembolism in a patient in need of such treatment by nasally administering an aqueous pharmaceutical formulation comprising the thrombin inhibitor HOOC—CH₂—(R)-Cgl-Aze-Pab (melagatran), or a pharmaceutically-acceptable derivative thereof.

An aqueous pharmaceutical formulation without a specific absorption enhancer present and comprising the thrombin inhibitor HOOC—CH₂—(R)-Cgl-Aze-Pab (melagatran), or a pharmaceutically-acceptable derivative thereof, for use by nasal administration in treating thromboembolism, is provided by the invention.

An aqueous pharmaceutical formulation containing the thrombin inhibitor HOOC—CH₂—(R)-Cgl-Aze-Pab (melagatran), or a pharmaceutically-acceptable derivative thereof, and other ingredients conventionally used in pharmaceutical formulations (but not including additional absorption enhancer components) for use by nasal administration in treating thromboembolism, is provided by the invention.

An aqueous pharmaceutical formulation containing the thrombin inhibitor HOOC—CH₂—(R)-Cgl-Aze-Pab (melagatran), or a pharmaceutically-acceptable derivative thereof, for use by nasal administration in treating thromboembolism, is provided by the invention.

The aqueous pharmaceutical formulations described herein are for use with all aspects of the invention, for example, the use and method of treatment aspects.

A preferred pH range of the formulation is in the range pH 3 to pH 8, particularly pH 4 to pH 7, and most especially pH 4 to pH 6.

A preferred pH range of the formulation suitable for nasal administration (for example to avoid or reduce irritation) is pH 4.5 to pH 6.5.

The dosage form used is preferably an aqueous solution of melagatran, prepared by known techniques, usually in which the active substance will constitute between 0.1 and 99% by weight of the preparation, more specifically between 0.1 and 50% by weight, particularly between 0.5 and 40% by weight, and more particularly between 5-20% (for example 50 and 200 mg/ml).

A preferred dose range of melagatran is from 1 mg to 9 mg melagatran in a volume for nasal administration of 5-400 μg/μL, more particularly 6-360 μg/μL, and most especially 25-150 μL.

A preferred patient for the nasal administration of the invention is a human patient.

The pharmaceutical formulations of the present invention comprising HOOC—CH₂—(R)Cgl-Aze-Pab, or a pharmaceutically-acceptable derivative thereof, are intended, primarily, for prophylaxis and treatment in arterial as well as venous thromboembolism. Other disease conditions in which thrombin inhibition is desirable are also provided for by the present invention, for example, inflammation and pulmonary fibrosis.

The term “inflammation” will be understood by those skilled in the art to include any condition characterised by a localised protective response elicited by injury or destruction of tissues resulting from any of the causes mentioned herein, and which is manifest by heat, swelling, pain, redness, dilation of blood vessels and/or increased blood flow, invasion of the affected area by white blood cells, loss of function and/or any other symptoms known to be associated with the inflammatory condition. The term will thus be understood to include inter alia acute, chronic, ulcerative, specific, allergic and necrotic inflammation, as well as all other forms of inflammation known to those skilled in the art.

Melagatran, and derivatives thereof, may thus be used in the direct treatment of inflammation resulting from injury, from viral or bacterial infection, or from a disease characterised by inflammation as one of its symptoms. Such diseases include autoimmune diseases, such as rheumatoid arthritis, psoriasis, allergy, asthma, rhinitis, pancreatitis, uticaria and inflammatory bowel syndrome.

However, melagatran, and derivatives thereof, are preferably used in the treatment of inflammation in patients with, or at risk of, a disease in which inhibition of thrombin is desired or required (see, for example, those listed in international patent application WO 97/23499), such as a thrombotic disease. Although the treatment may be of patients whose inflammatory and thrombotic diseases are unrelated, we prefer that the treatment is of a patient with a thrombotic disease in which inflammation plays a part in triggering coagulation. For example, inflammation may arise in blood vessel walls due to the presence and/or the action of microbes and/or the agents released thereby, physical damage, atheroscelorotic lesions and other inflammation-inducing agents. It is preferred that melagatran, and derivatives thereof, are used in the treatment of inflammation in patients having, or at risk of having, a thrombus.

For the avoidance of doubt, as used herein, the term “treatment” includes the therapeutic and/or prophylactic treatment of inflammation.

The term “pulmonary fibrosis” (PF) will be understood by those skilled in the art to include any condition characterised by one or more of (a) collagen deposition in the lung, (b) scarring (fibrosis) of the lung (including the alveoli and in the interstitium), and/or (c) regions of severe thickening of the alveolar walls, one or more of which may result in a chronic stiffness in the lungs and/or a decreased ability of the lung tissue to transport oxygen.

The PF may be a secondary fibrosis, which may be brought on by an inflammatory condition, such as sarcoidosis, rheumatoid arthritis, systemic sclerosis, scleroderma, extrinsic allergic alveolitis, severe asthma, systemic granulomatosis vasculitis and/or adult respiratory distress syndrome (ARDS), or it may be “idiopathic” PF (IPF).

The term “IPF” will be understood to include any form of PF where the underlying causes of the condition are unknown and/or to include the definition provided in the consensus statement in Am. J. Respir. Crit. Care Med., 161, 646 (2000), the relevant disclosure in which document is hereby incorporated by reference.

Particular forms of IPF that may be mentioned include inter alia desquamative interstitial pneumonitis (DIP), acute interstitial pneumonia (AIP), non-specific interstitial pneumonia (NSIP), respiratory bronchiolitis-associated interstitial lung disease (RBILD), bronchiolitis obliterans organising pneumonia (BOOP), lymphoid interstitital pneumonia (LIP) and, particularly, usual interstitial pneumonitis (UIP) (see, for example, Am. J. Respit. Crit. Care Med., 157, 1301 (1998)).

Also provided by the invention is a process for the manufacturing of a pharmaceutical formulation, for use according to the invention, comprising forming an aqueous solution of the therapeutically active compound HOOC—CH₂—(R)Cgl-Aze-Pab, or a pharmaceutically-acceptable derivative thereof, optionally adjusting the pH (optionally using a buffering agent) to a therapeutically acceptable pH and isotonicity, for instance between 3 to 8, preferably between 4 and 7 or 4 and 6, for example pH 5, and mixing all ingredients. The pH can be adjusted by adding e.g. HCl or NaOH.

The formulations of the present invention are free of additional absorption enhancer components, although other ingredients conventionally used in pharmaceutical formulations such as buffers such as K₂HPO₄: Na₂HPO₄, carriers, thickening and precipitation agents and isotonic agents such as NaCl known by a skilled person in the art may also be added to a pharmaceutical formulation of the present invention. Pharmaceutically-acceptable solvents other than water may also be used if suitable for nasal administration.

“Pharmaceutically-acceptable derivatives” of melagatran includes salts (e.g. pharmaceutically-acceptable non-toxic organic or inorganic acid addition salts) and solvates. It will be appreciated that the term further includes derivatives that have, or provide for, the same biological function and/or activity as melagatran. Thus, for the purposes of this invention, the term also includes prodrugs of melagatran (such as ximelagatran). “Prodrugs” of melagatran include any composition of matter that, following administration, is metabolised in vivo to form melagatran in an experimentally-detectable amount, and within a predetermined time (e.g. within a dosing interval of between 6 and 24 hours (i.e. once to four times daily)). Formulations comprising pharmaceutically-acceptable derivatives of melagatran may be prepared for use within a pre-determined period of time, for example for immediate use, or for use within 2 to 6 hours.

Thrombin inhibitors other than melagatran, or a pharmaceutically-acceptable derivative thereof, may also be used in the invention.

The following description is illustrative, but not limiting, of aspects of the invention.

### EXPERIMENTAL PART

Use of Absorption Enhancers

Using standard techniques, melagatran was administered to rats in formulations with and without the enhancers SDS (sodium dodecyl sulfate) or EDTA. The bioavailability was measured by analysis of blood plasma samples using standrad techniques.

The bioavailability of intranasal melagatran 20 μmol/kg alone was approximately 10%, with enhancers improving this result up to about 19%.

Nasal Administration of Melagatran in Healthy Male Humans

The rate and extent of absorption of melagatran as well as the safety and tolerability were investigated after intranasal administration to six healthy male subjects (between 20 and 40 years of age, body weight between 66 and 86 kg). The trial comprised three study days, separated by wash-out periods of 6-28 days. On study day 1, a single dose of 5 mg of melagatran was administered. The following two study days 10 mg and 20 mg, respectively, were administered.

Samples for determination of plasma concentration of melagatran (by LC-MS) and for degree of anticoagulation were collected before and up to 10 hours after drug administration. Safety measurements included blood pressure, heart rate and recording of adverse events.

The absorption of melagatran after intranasal administration was rapid and the median bioavailabilities for the three dose levels, 5 mg, 10 mg and 20 mg, were 19%, 12% and 19%, respectively. The bioavailability of melagatran for the six subjects at the three doses ranged from 7% to 45%. There was no indication of a dose dependent rate or extent of absorption. The safety and tolerability of melagatran when administered nasally were considered satisfactory.

Preparation of Melagatran Test Compositions

Melagatran was dissolved in water and the composition adjusted to a pharmaceutically-acceptable isotonicity and pH (such as pH 5). The solution was aseptically filled into a glass bottle and a pump and applicator fitted (to give a metered dose of 50 microlitres).

Melagatran Liquid Nasal Spray 50 mg/ml, Glass Spray Bottle Containing 5 ml

| INGREDIENT | FORMULA (mg/ml) |
|---|---|
| Melagatran | 50 |
| Hydrochloric acid for adjustment to pH 5 | q.s. |
| Water purified | to 1 ml |

Melagatran Liquid Nasal Spray 200 mg/ml, Glass Spray Bottle Containing 5 ml

| INGREDIENT | FORMULA (mg/ml) |
|---|---|
| Melagatran | 200 |
| Hydrochloric acid for adjustment to pH 5 | q.s. |
| Water purified | to 1 ml |

Abbreviations
Aze=(S)-Azetidine-2-carboxylic acid; Cgl=(S)-Cyclohexyl glycine; Pab=1-Amidino-4-aminomethyl benzene.

1. An aqueous pharmaceutical formulation for nasal administration, comprising the thrombin inhibitor HOOC—CH₂—(R)-Cgl-Aze-Pab (melagatran), or a pharmaceutically acceptable derivative thereof.

2-4. (canceled)

5. A method of treating a patient in need of antithrombotic treatment comprising nasally administering an aqueous pharmaceutical formulation comprising the thrombin inhibitor HOOC—CH₂—(R)-Cgl-Aze-Pab (melagatran), or a pharmaceutically acceptable derivative thereof.

6. A method of treating thromboembolism in a patient in need of such treatment comprising nasally administering an aqueous pharmaceutical formulation comprising the thrombin inhibitor HOOC—CH₂—(R)-Cgl-Aze-Pab (melagatran), or a pharmaceutically acceptable derivative thereof.
